(19) United States
(12) Patent Application Publication
(10) Pub. No.: US 2020/0141957 A1
(43) Pub. Date: May 7, 2020

(54) IDENTIFYING STATUS OF MALE FERTILITY BY DETERMINING SPERM CAPACITATION AND COMPANION COLLECTION KIT

(71) Applicant: Androvia LifeSciences, LLC, Mountainside, NJ (US)

(72) Inventors: G. Charles Ostermeier, Gillette, NJ (US); Alexander Travis, Ithaca, NY (US)

(73) Assignee: Androvia LifeSciences, LLC, Mountainside, NJ (US)

(21) Appl. No.: 16/672,056

(22) Filed: Nov. 1, 2019

Related U.S. Application Data
(60) Provisional application No. 62/884,785, filed on Aug. 9, 2019.
(60) Provisional application No. 62/755,087, filed on Nov. 2, 2018.

(51) Int. Cl. G01N 33/92; G01N 1/42

(52) U.S. Cl. CPC G01N 33/92; G01N 2405/10; G01N 1/42

(57) ABSTRACT

A method for identifying fertility status of a human male using an extended sperm sample stored for a period of greater than 2 hours is described herein.

### CROSS REFERENCE TO RELATED APPLICATIONS

This application claims priority to U.S. provisional application No. 62/884,785, filed Aug. 9, 2019, and U.S. provisional application No. 62/755,087, filed Nov. 2, 2018, each of which is herein incorporated by reference in its entirety.

### FIELD OF THE INVENTION

This invention relates generally to the field of male fertility and more specifically to determining male fertility status based on distribution patterns of the ganglioside, G_{M1}, following sperm capacitation from sperm samples stored for greater than 2 hours in a medium comprising an extender solution maintained at a temperature ranging from about 4° C. to about 25° C., and a companion collection kit for use with the Cap-Score™ assay.

### BACKGROUND OF THE INVENTION

In the US, ten percent of couples have medical appointments related to infertility with about 40% to about 50% of infertility being associated with the male. Globally, this translates to over 73 million infertile couples. Typical male reproductive health exams assess sperm number, appearance, and motility. Unfortunately, more than half of infertile men have sperm that meet normal parameters for these descriptive criteria and are only identified as having “idiopathic infertility” after repeatedly failing at both natural conception and techniques of assisted reproduction such as intra-uterine insemination (IUI). Because each failed cycle inflicts great physical, emotional, and financial tolls on couples and it costs the US healthcare system over $5 billion annually, there is a tremendous need for a practical test of sperm function (Babigumira et al., Journal of Assisted Reproduction and Genetics, 2018, 35(1): 99-106). Such tests would allow clinicians to direct their patients toward a technology of assisted reproduction that would give them the best chance of generating a pregnancy (Cardona et al., Molecular Reproduction and Development, 2017, 84(5): 423-435; Schinfeld et al., Molecular Reproduction and Development 2018, 85(8-9): 654-664).

Upon entrance into the female tract, sperm are not immediately able to fertilize an egg. Rather, they must undergo a process of functional maturation known as “capacitation.” This process relies upon their ability to respond to specific stimuli by having specific changes in their cell membrane, namely a change in the distribution pattern of the ganglioside G_{M1} in response to exposure to stimuli for capacitation.

The Cap-Score™ assay is used to assess male fertility by measuring differences in certain G_{M1} distribution patterns in a sperm sample. In performing the Cap-Score™ assay, the sperm sample was typically freshly prepared, kept warm at body temperature and transported to the testing lab within about 30 minutes. When performing the Cap-Score™ assay, the sperm sample was not stored for any extended period longer than 2 hours (supra, Cardona et al., 2017).

### SUMMARY OF THE INVENTION

The present invention provides for methods for extending the sperm samples and methods of extending the storage time of the sperm sample prior to preparing it for the Cap-Score™ assay. More specifically, the present invention improves tolerance of the sperm cell to capacitate when stored under certain conditions for at least 2 hours or more, thus enabling a patient (or doctor) to collect the sperm sample and ship the sperm sample to a laboratory that can perform the Cap-Score™ assay. The convenience of home collection of a sperm sample greatly improves the Cap-Score™ assay availability without loss of accuracy or efficiency.

In an embodiment of the invention, this disclosure provides a method for identifying male fertility status.

In one embodiment, the method for identifying the fertility status of a human male comprises the steps: a) obtaining a sperm sample from a human male; b) introducing into the sperm sample a fixed volume of a semen extender solution at a dilution volume ratio to give an extended sperm sample; c) maintaining the extended sperm sample at a temperature range of about 4° C. to about 25° C. for a time span of greater than 2 hours; d) performing a Cap-Score™ assay on the extended sperm sample of step (c) to determine the percentage of sperm capable of undergoing capacitation (Cap-Score™); and e) determining fertility status of the human male. In one embodiment, the resulting Cap-Score™ for the extended sperm sample from the Cap-Score™ assay is not significantly different from a Cap-Score™ resulting from a similarly processed fresh sample from the same individual.

In one embodiment, the method for identifying fertility status of a human male comprises maintaining the temperature of the extended sperm sample in a range from about 4° C. to about 25° C. for greater than 2 hours. In some embodiments, the method for identifying fertility status of a human male comprises maintaining the temperature of the extended sperm sample in a range from about 4° C. to about 10° C. for greater than 2 hours.

In some embodiments, the extended time span for storage of the sperm sample is about 2 hours to about 24 hours. In some embodiments, the extended time span for the sperm sample in shipment is about 12 hours to about 48 hours. In some embodiments, the extended time span for the sperm sample in shipment is at least 12 hours. In some embodiments, the extended time span for the sperm sample in shipment is at least 18 hours.

In some embodiments, the semen extender comprises a buffer system. Any buffering system known in the art is useful in the present invention. In some embodiments, the semen extender comprises a buffer selected from the group consisting of bicarbonate buffer, citrate buffer, hydroxymethylaminomethane (TRIS) buffer, TRIS/citric acid buffer, TRIS/citrate buffer, 4-(2-hydroxyethyl)-1-piperazineethanesulfonic acid (HEPES) buffer, HEPES/TRIS buffer, N′-tris (hydroxymethyl)methyl-2-aminoethane (TES) and hydroxymethylaminomethane (TRIS) buffer (TES/TRIS buffer), and combination thereof. In some embodiments, the buffer comprises TES/TRIS buffer (or “TEST” buffer).

In some embodiments, the semen extender comprises at least one protein. In some embodiments, the protein is selected from the group consisting of albumin, equine serum, bovine serum, soy protein, fetal cord serum ultrafiltrate, plasmanate, egg yolk, skim milk, lipoproteins, fatty acid binding proteins, and combinations thereof. In some embodiments, the semen extender solution comprises a protein selected from the group consisting of egg yolk, milk protein, and combinations thereof. In an embodiment, the semen extender solution comprises egg yolk.

In some embodiments, the semen extender further comprises an antibiotic. In some embodiments, the antibiotic is selected from the group consisting of gentamicin, penicillin, streptomycin, amphotericin, and combinations thereof. In an embodiment, the antibiotic is gentamicin.

In some embodiments, the method comprises performing one or more additional steps prior to performing the Cap-Score™ assay. In some embodiments, the additional step comprises a step selected from centrifuging the extended semen sample to separate the seminal plasma from the sperm cells and to collect sperm cell pellets, resuspending the sperm cell pellets into a medium (e.g. mHTF) with one or more capacitation stimuli (Cap) and, optionally, a medium (e.g. mHTF) without capacitation stimuli (non-Cap), and incubating the resulting cell suspensions in Cap and, optionally, non-Cap media for 3 hours at 37° C.

In some embodiments, the dilution volume ratio for the sperm sample to the semen extender solution is about 10:1 to about 1:10. In some embodiments, the dilution volume ratio for the sperm sample to the semen extender solution is about 1:10. In some embodiments, the dilution volume ratio for the sperm sample to the semen extender solution is about 10:1. In some embodiments, the dilution volume ratio for the sperm sample to the semen extender solution is about 1:1.

In some embodiments, the sperm sample is stored in a plastic tube with a conical bottom and seal cap. In some embodiments, the plastic tube is selected from the group consisting of polypropylene, polystyrene, polyethylene terephthalate (PET), low-density polyethylene (LDPE), polyallomer (PA), and polycarbonate (PC).

In some embodiments, the method further comprises using a sperm sample collection kit that includes one or more of: sperm storage containers, transfer pipettes, an insulating pouch, a cold pack, a thermally insulating container, and a semen extender solution.

In an embodiment, this disclosure provides a method for identifying fertility status of a human male individual comprising the steps: a) obtaining a sperm sample from a human male; b) introducing into the sperm sample a fixed volume of a semen extender solution at a dilution volume ratio to give an extended sperm sample; c) cooling the extended sperm sample of step (b) to reach a temperature ranging from about 4° C. to about 10° C. inside a thermally insulating container to provide a chilled extended sperm sample, d) maintaining the chilled extended sperm sample at a temperature range of about 4° C. to about 25° C. for an extended time span; wherein the temperature is maintained by packing the extended sperm sample in a thermal insulating pouch with a cold pack chilled to a defined temperature; and e) performing Cap-Score™ assay (determining of the distribution of G_{M1} patterns that resulted from capacitation) on the extended sperm sample of step (d) and determining fertility status of the human male.

In some embodiments, the cooling step (c) takes place over a period of about one hour. In some embodiments, the extended time span in step (d) is at least 18 hours. In some embodiments, the cold pack is chilled to 4° C. In some embodiments, the cold pack is chilled to −20° C. In some embodiments, the method further comprises using a companion sperm sample collection kit that includes one or more of: sperm storage containers, transfer pipettes, an insulating pouch, a cold pack, a thermally insulating container, and a semen extender solution.

In an embodiment, this invention provides a kit for determination of male fertility status comprising one or more of: sperm storage containers, transfer pipettes, an insulating pouch, a cold pack, a thermally insulating container, a semen extender solution, an agent for stimulating capacitation, capacitating media, non-capacitating media, fixative reagents, and reagents for determining the distribution of G_{M1} patterns.

### BRIEF DESCRIPTION OF THE DRAWINGS

FIG. 1 is a flow chart that illustrates semen sample preparation and a timeline.

FIG. 2, comprising FIGS. 2A-2E, illustrates the transportation of the extended human raw ejaculate using a home collection kit comprising a cold pack chilled to 4° C.

FIG. 3 illustrates the temperature profile produced by a cold pack chilled to 4° C. in a sealed insulated Styrofoam container as measured over 24 hours.

FIG. 4, comprising FIGS. 4A-4D, illustrates the monitoring the temperature profile for the semen samples maintained in refrigerator at 4° C.

FIG. 5 illustrates the temperature profile for samples maintained in the refrigerator at 4° C. over 24 hours.

FIG. 6 illustrates Cap-Score™ test results for samples stored with cold pack chilled to 4° C. in a sealed insulated Styrofoam container.

FIG. 7 illustrates Cap-Score™ test results for samples maintained in a refrigerator at 4° C.

FIG. 8, comprising FIGS. 8A-8E, illustrates the transportation of the extended human raw ejaculate using a home collection kit comprising a cold pack chilled to −20° C.

FIG. 9 illustrates the temperature profile produced by cold pack chilled to −20° C. in a sealed insulated Styrofoam container as measured over 24 hours.

### DETAILED DESCRIPTION OF THE INVENTION

The present disclosure is based on the observations that certain G_{M1} distribution patterns can provide information regarding male fertility status. Determination of G_{M1} patterns is described in, for example, U.S. Pat. Nos. 7,160,676, 7,670,763, and 8,367,313, and in US Publication No. 20170248584, the disclosures of which are incorporated herein by reference. The Cap-Score™ assay is based on a change in the frequency of certain G_{M1} distribution patterns upon exposure to capacitating stimuli. The present disclosure provides methods for determination of male fertility status and companion collection/transporting kits. The companion kit includes a semen extender solution for preserving sperm cell viability during sperm storage and transporting for a duration greater than 2 hours.

In the present disclosure, the unexpected finding that semen can be collected in a private setting, such as in a patient's home, and prepared and stored for overnight shipping without significant degradation and without significant variation in the resulting Cap-Score™, is shown.

### Definitions

As used in the preceding sections and throughout the rest of this specification, unless defined otherwise, all technical and scientific terms used herein have the same meaning as is commonly understood by one of skill in the art to which this invention belongs. All patents and publications referred to herein are incorporated by reference in their entireties.

The term “capacitating” (Cap) conditions as used herein, generally refers to conditions under which sperm cells have been incubated with one or more of the stimuli for capacitation. Specifically, this requires the presence of bicarbonate and/or calcium ions in the medium, and the presence of a sterol acceptor such as serum albumin or a cyclodextrin. Sperm that have successfully responded to capacitating conditions have acquired the ability to undergo acrosome exocytosis and have acquired a hyperactivated pattern of motility.

The term “non-capacitating” (non-Cap) conditions as used herein, refers to conditions under which sperm cells are not incubated with one or more stimuli for capacitation. Sperm that have not been exposed to capacitating conditions, or have been exposed but have not responded to those capacitating conditions, do not undergo acrosome exocytosis induced by a physiological ligand such as the zona pellucida, solubilized proteins from the zona pellucida, or progesterone. In addition, such sperm will not demonstrate hyperactivated motility.

The term “Cap-Score™ assay” as used herein, generally refers to a method for identifying fertility status of a human male individual comprising the steps: (1) staining a fixed, capacitated, and optionally, a fixed, non-capacitated sperm sample from the same individual for G_{M1}; (2) imaging the stained fixed sperm samples to determine a frequency of selected G_{M1} patterns in the capacitated sperm and, optionally the non-capacitated sperm; and (3) comparing the frequency of selected G_{M1} patterns that are associated with sperm responding to capacitation conditions to the total frequency of G_{M1} patterns in sperm, and comparing that value to a reference value in a known, fertile population. The test provides an analysis of sperm on a molecular level to determine the percentage of sperm capable of undergoing capacitation (Cap-Score™).

In some embodiments, a method for identifying fertility status of a male is described. In some embodiments, the method comprises obtaining a sperm sample from an individual. In some embodiments, the sperm sample is obtained using a home collection kit as described in more detail herein. In some embodiments, the sperm sample is collected by the individual in an at-home setting. In some embodiments, the sperm sample is collected in a doctor's office, in a laboratory, or in a clinic.

In some embodiments, the semen sample is stored in a container with a closeable lid before performing the Cap-Score™ assay. In some embodiments, the container may be plastic, glass, or other durable material. In some embodiments, the container is plastic. In some embodiments, the container comprises plastic selected from the group consisting of polypropylene, polystyrene, polyethylene terephthalate (PET), low-density polyethylene (LDPE), polyallomer (PA), and polycarbonate (PC).

In some embodiments, the sperm sample is combined with a semen extender.

In some embodiments, use of a semen extender is employed to extend the viability of the sperm sample. In some embodiments, the semen extender is any commercially available or subsequently developed semen extender solution. A semen extender represents an osmotically balanced salt solution, and may comprise one or more of an energy source (e.g., sugars such as glucose, fructose, dextrose, sucrose, sorbitol, pyruvate), lipids (e.g., soy lecithin, egg yolk lipids, milk lipids), a protein source (e.g., caseins, albumins, seminal plasma), antibiotics (e.g., gentamicin, penicillin, streptomycin, amphotericin), a buffering system and/or electrolytes (e.g., balanced and isotonic electrolyte solution, TRIS, TES, TES/TRIS (TEST), citric acid, sodium citrate, HEPES). In some embodiments, the semen extender comprises egg yolk, glucose and citrate.

In some embodiments, the semen extender comprises one or more proteins or lipids for use as a cold shock protectant. In some embodiments, the semen extender comprises a protein selected from the group consisting of albumin, equine serum, bovine serum, soy protein, soy lecithin, fetal cord serum ultrafiltrate, plasmanate, egg yolk, egg yolk lecithin, skim milk, casein, lipoproteins, fatty acid binding proteins, and combinations thereof. In some embodiments, the semen extender comprises a protein selected from the group consisting of egg yolk, soy protein, milk, and combinations thereof. In some embodiments, the protein comprises egg yolk. In some embodiments, the protein comprises chicken egg yolk. The main component in the egg yolk plasma is low-density lipoproteins (LDL). The LDL in the egg yolk have a liquid lipid core surrounded by phospholipids. In some embodiments, the protein comprises LDL.

In some embodiments, the protein comprises whole egg yolk. In some embodiments, the protein comprises a fraction of the whole egg yolk, for example, clarified egg yolk prepared by centrifugation of whole egg yolk, or low-density-proteins prepared by extraction of LDL fraction from the egg yolk plasma.

In some embodiments, the semen extender comprises a phospholipid derived from an animal source, e.g. phospholipids of the LDL (egg yolk lecithin). In some embodiments, the semen extender comprises a phospholipid derived from the non-animal source, e.g. soy-lecithin.

In some embodiments, the semen extender may be biologically buffered so that it exhibits a pH that maintains the viability of the cells. In some embodiments, the semen extender comprises a buffer to maintain a pH of between about 6.9 and about 7.5 in the semen extender. In some embodiments, the pH is maintained at about 6.9, 7.0, 7.1, 7.2, 7.3, 7.4, or 7.5. Commonly used biological buffers are available under the names including TRIS, HEPES, and TES. The amount of biological buffer provided in the extender depends on the strength of the biological buffer and the desired buffering capacity of the extender, for example, concentrations of commonly used buffers may include: about 10 mM bicarbonate, 20-25 mM HEPES, or about 20-25 mM TRIS-HCl.

In some embodiments, the semen extender comprises a buffer selected from the group consisting of bicarbonate buffer, citrate buffer, sodium citrate buffer, hydroxymethylaminomethane (TRIS) buffer, TRIS/citric acid buffer, TRIS/citrate buffer, 4-(2-hydroxyethyl)-1-piperazineethanesulfonic acid (HEPES) buffer, HEPES/TRIS buffer, N′-tris (hydroxymethyl)methyl-2-aminoethane (TES) and hydroxymethylaminomethane (TRIS) buffer (TES/TRIS buffer), tris(hydroxymethyl)methylamino]propanesulfonic acid (TAPS) buffer, 2-(bis(2-hydroxyethyl)amino)acetic acid (bicine) buffer, 3-[N-tris(hydroxymethyl)methylamino]-2-hydroxypropanesulfonic acid (tricine) buffer, 3-[N-tris(hydroxymethyl)methylamino]-2-hydroxypropanesulfonic acid (TAPSO) buffer, 3-(N-morpholino)propanesulfonic acid (MOPS) buffer, Piperazine-N,N′-bis(2-ethanesulfonic acid) (PIPES) buffer, 2-(N-morpholino)ethanesulfonic acid (MES) buffer, and combinations thereof.

In some embodiments, the semen extender is an egg yolk based extender. In some embodiments, the egg yolk based extender may contain 16.0% to 24.0% by volume of egg yolk and 45.0% to 70.0% by volume of a buffer solution. In some embodiments, the egg yolk based extender comprises whole chicken egg yolk. In some embodiments, the egg yolk based extender may contain 16.0%, 20.0%, or 24.0% by volume of egg yolk.

In some embodiments, the semen extender comprises egg yolk and citrate buffer. In some embodiments, the egg yolk based extender comprises egg yolk and TRIS buffer. In some embodiments, the egg yolk based extender comprises egg yolk and TRIS/citrate buffer. In some embodiments, the egg yolk based extender comprises egg yolk and TRIS/citric acid buffer. In some embodiments, the semen extender comprises whole chicken egg yolk and citrate buffer. In some embodiments, the egg yolk based extender comprises whole chicken egg yolk and TRIS buffer. In some embodiments, the egg yolk based extender comprises whole chicken egg yolk and TRIS/citrate buffer. In some embodiments, the egg yolk based extender comprises whole chicken egg yolk and TRIS/citric acid buffer.

In some embodiments, the semen extender exhibits an osmolality of about 250 mOsM to about 350 mOsM. In some embodiments, the semen extender exhibits an osmolality of about 290 mOsM to about 320 mOsM. In some embodiments, the semen extender comprises at least about 90 wt. % water.

In some embodiments, the semen extender may include an energy source, such as carbohydrates, for providing energy for the sperm cells. In some embodiments, the carbohydrate is a simple sugar. In some embodiments, the carbohydrate is selected from the group consisting of sorbitol, fructose, sucrose, dextrose, glucose, and lactose. In some embodiments, the carbohydrates may be used alone or in combination with one or more other energy sources. In an embodiment of the invention, the carbohydrate is present in an amount sufficient to provide the cells with energy. In one embodiment, the amount of glucose as a carbohydrate source is about 0.09 g/L to about 1.8 g/L. In some embodiments, the amount of glucose as a carbohydrate source is about 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 g/L.

In some embodiments, the semen extender comprises an antibiotic. In some embodiments, the antibiotics is a compound selected from the group consisting of penicillins, tetracyclines, cephalosporins, lincomycins, macrolides, glycopeptides, aminoglycosides, carbapenems, and combinations thereof. In some embodiments, the antibiotic is selected from the group consisting of gentamicin, tobramycin, amikacin, penicillin, streptomycin, amoxicillin, doxycycline, minocycline, tetracycline, eravacycline, cephalexin, clindamycin, lincomycin, clarithromycin, erythromycin, metronidazole, azithromycin, levofloxacin, moxifloxacin, cefuroxime, ceftriaxone, cefdinir, dalbavancin, oritavancin, telavancin, vancomycin, ertapenem, doripenem, meropenem, imipenem/cilastatin, bacitracin, neomycin, polymyxin B, amphotericin, and combinations thereof. In some embodiments, the antibiotic is gentamicin.

In some embodiments, the semen extender is chosen for its ability to maintain viability and motility of the initial sperm sample. In some embodiments, the viability of the sperm sample is at least 40% of the initial viability, and the motility corresponds to a level of at least 40% of the initial motility. In some embodiments, the levels of viability and motility are at least about 70% of the initial values. In some embodiments, the components of the semen extender composition provided in the home collection kit according to the invention can be selected to help maintain viability and motility of the sperm cells.

Some semen extenders that may be useful in the present invention include (1) CYB medium (Weidel et al., J. Androl. 8: 41-47 (1987)) and (2) Refrigeration medium—TYB with gentamicin (Irvine Scientific, Santa Ana, Calif.; catalogue #90129).

In some embodiments, the semen extender may include components derived from an animal source and/or the semen extender composition can include components derived from a non-animal source. In some embodiments, the semen extender may be characterized as substantially free of components derived from an animal source. It should be understood that “substantially free” means the extender contains less than about 0.1 wt. % of a component derived from an animal source. It should be understood that the characterization of the semen extender as including or not including components derived from an animal source is not meant to reflect the semen or ejaculate which is added to the semen extender composition.

In some embodiments, the semen extender may be free of any component derived from an animal source. In some embodiments, the semen extender composition may include a component or components that are derived from an animal and these components can be present in the semen extender composition in amounts greater than 0.1 wt. % based on the weight of the semen extender composition.

In some embodiments, the sperm sample is introduced into the semen extender solution at a dilution volume ratio of the sperm sample to the semen extender solution of about 10:1 to about 1:10. In some embodiments, the dilution volume ratio of the sperm sample to the semen extender solution is about 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10. In some embodiments, the dilution volume ratio of the sperm sample to the semen extender solution is about 3:1, 2:1 or 1:1. In some embodiments, the dilution volume ratio of the sperm sample to the semen extender solution is about 1:10. In some embodiments, the dilution volume ratio of the sperm sample to the semen extender solution is about 1:10. In some embodiments, the dilution volume ratio of the sperm sample to the semen extender solution is about 1:1.

In some embodiments, the sample is maintained at a temperature range selected from the group consisting of about 1° C. to about 25° C., about 1° C. to about 24° C., about 1° C. to about 23° C., about 1° C. to about 22° C., about 1° C. to about 21° C., about 1° C. to about 20° C., about 1° C. to about 19° C., about 1° C. to about 18° C., about 1° C. to about 17° C., about 1° C. to about 16° C., about 1° C. to about 15° C., about 1° C. to about 14° C., about 1° C. to about 13° C., about 1° C. to about 12° C., about 1° C. to about 11° C., about 1° C. to about 10° C., about 1° C. to about 9° C., about 1° C. to about 8° C., about 1° C. to about 7° C., about 1° C. to about 6° C., about 1° C. to about 5° C., about 1° C. to about 4° C., about 1° C. to about 3° C., and about 1° C. to about 2° C.

In some embodiments, the sample is maintained at a temperature range selected from the group consisting of about 2° C. to about 25° C., about 2° C. to about 24° C., about 2° C. to about 23° C., about 2° C. to about 22° C., about 2° C. to about 21° C., about 2° C. to about 20° C., about 2° C. to about 19° C., about 2° C. to about 18° C., about 2° C. to about 17° C., about 2° C. to about 16° C., about 2° C. to about 15° C., about 2° C. to about 14° C., about 2° C. to about 13° C., about 2° C. to about 12° C., about 2° C. to about 11° C., about 2° C. to about 10° C., about 2° C. to about 9° C., about 2° C. to about 8° C., about 2° C. to about 7° C., about 2° C. to about 6° C., about 2° C. to about 5° C., about 2° C. to about 4° C., and about 2° C. to about 3° C.

In some embodiments, the sample is maintained at a temperature range selected from the group consisting of about 3° C. to about 25° C., about 3° C. to about 24° C., about 3° C. to about 23° C., about 3° C. to about 22° C., about 3° C. to about 21° C., about 3° C. to about 20° C., about 3° C. to about 19° C., about 3° C. to about 18° C., about 3° C. to about 17° C., about 3° C. to about 16° C., about 3° C. to about 15° C., about 3° C. to about 14° C., about 3° C. to about 13° C., about 3° C. to about 12° C., about 3° C. to about 11° C., about 3° C. to about 10° C., about 3° C. to about 9° C., about 3° C. to about 8° C., about 3° C. to about 7° C., about 3° C. to about 6° C., about 3° C. to about 5° C., and about 3° C. to about 4° C.

In some embodiments, the sample is maintained at a temperature range selected from the group consisting of about 4° C. to about 25° C., about 4° C. to about 24° C., about 4° C. to about 23° C., about 4° C. to about 22° C., about 4° C. to about 21° C., about 4° C. to about 20° C., about 4° C. to about 19° C., about 4° C. to about 18° C., about 4° C. to about 17° C., about 4° C. to about 16° C., about 4° C. to about 15° C., about 4° C. to about 14° C., about 4° C. to about 13° C., about 4° C. to about 12° C., about 4° C. to about 11° C., about 4° C. to about 10° C., about 4° C. to about 9° C., about 4° C. to about 8° C., about 4° C. to about 7° C., about 4° C. to about 6° C., and about 4° C. to about 5° C.

In some embodiments, the sample is maintained at a temperature range selected from the group consisting of about 5° C. to about 25° C., about 5° C. to about 24° C., about 5° C. to about 23° C., about 5° C. to about 22° C., about 5° C. to about 21° C., about 5° C. to about 20° C., about 5° C. to about 19° C., about 5° C. to about 18° C., about 5° C. to about 17° C., about 5° C. to about 16° C., about 5° C. to about 15° C., about 5° C. to about 14° C., about 5° C. to about 13° C., about 5° C. to about 12° C., about 5° C. to about 11° C., about 5° C. to about 10° C., about 5° C. to about 9° C., about 5° C. to about 8° C., about 5° C. to about 7° C., and about 5° C. to about 6° C.

In an embodiment of the invention, the extended sperm sample is maintained at a temperature from about 6° C. to about 20° C. In some embodiments, the sample is maintained at a temperature range selected from the group consisting of about 6° C. to about 25° C., about 6° C. to about 24° C., about 6° C. to about 23° C., about 6° C. to about 22° C., about 6° C. to about 21° C., about 6° C. to about 20° C., about 6° C. to about 19° C., about 6° C. to about 18° C., about 6° C. to about 17° C., about 6° C. to about 16° C., about 6° C. to about 15° C., about 6° C. to about 14° C., about 6° C. to about 13° C., about 6° C. to about 12° C., about 6° C. to about 11° C., about 6° C. to about 10° C., about 6° C. to about 9° C., about 6° C. to about 8° C., and about 6° C. to about 7° C.

In some embodiments, the sample is maintained at a temperature range selected from about 7° C. to about 25° C., about 7° C. to about 24° C., about 7° C. to about 23° C., about 7° C. to about 22° C., about 7° C. to about 21° C., about 7° C. to about 20° C., about 7° C. to about 19° C., about 7° C. to about 18° C., about 7° C. to about 17° C., about 7° C. to about 16° C., about 7° C. to about 15° C., about 7° C. to about 14° C., about 7° C. to about 13° C., about 7° C. to about 12° C., about 7° C. to about 11° C., about 7° C. to about 10° C., about 7° C. to about 9° C., and about 7° C. to about 8° C.

In some embodiments, the sample is maintained at a temperature range selected from the group consisting of about 8° C. to about 25° C., about 8° C. to about 24° C., about 8° C. to about 23° C., about 8° C. to about 22° C., about 8° C. to about 21° C., about 8° C. to about 20° C., about 8° C. to about 19° C., about 8° C. to about 18° C., about 8° C. to about 17° C., about 8° C. to about 16° C., about 8° C. to about 15° C., about 8° C. to about 14° C., about 8° C. to about 13° C., about 8° C. to about 12° C., about 8° C. to about 11° C., about 8° C. to about 10° C., and about 8° C. to about 9° C.

In some embodiments, the sample is maintained at a temperature range selected from the group consisting of about 9° C. to about 25° C., about 9° C. to about 24° C., about 9° C. to about 23° C., about 9° C. to about 22° C., about 9° C. to about 21° C., about 9° C. to about 20° C., about 9° C. to about 19° C., about 9° C. to about 18° C., about 9° C. to about 17° C., about 9° C. to about 16° C., about 9° C. to about 15° C., about 9° C. to about 14° C., about 9° C. to about 13° C., about 9° C. to about 12° C., about 9° C. to about 11° C., and about 9° C. to about 10° C.

In some embodiments, the sample is maintained at a temperature range selected from the group consisting of about 10° C. to about 25° C., about 10° C. to about 24° C., about 10° C. to about 23° C., about 10° C. to about 22° C., about 10° C. to about 21° C., about 10° C. to about 20° C., about 10° C. to about 19° C., about 10° C. to about 18° C., about 10° C. to about 17° C., about 10° C. to about 16° C., about 10° C. to about 15° C., about 10° C. to about 14° C., about 10° C. to about 13° C., about 10° C. to about 12° C., and about 10° C. to about 11° C.

In some embodiments, the sample is maintained at a temperature range selected from the group consisting of about 11° C. to about 25° C., about 11° C. to about 24° C., about 11° C. to about 23° C., about 11° C. to about 22° C., about 11° C. to about 21° C., about 11° C. to about 20° C., about 11° C. to about 19° C., about 11° C. to about 18° C., about 11° C. to about 17° C., about 11° C. to about 16° C., about 11° C. to about 15° C., about 11° C. to about 14° C., about 11° C. to about 13° C., and about 11° C. to about 12° C. In some embodiments, the sample is maintained at a temperature range selected from the group consisting of about 12° C. to about 25° C., about 12° C. to about 24° C., about 12° C. to about 23° C., about 12° C. to about 22° C., about 12° C. to about 21° C., about 12° C. to about 20° C., about 12° C. to about 19° C., about 12° C. to about 18° C., about 12° C. to about 17° C., about 12° C. to about 16° C., about 12° C. to about 15° C., about 12° C. to about 14° C., and about 12° C. to about 13° C. In some embodiments, the sample is maintained at a temperature range selected from the group consisting of about 13° C. to about 25° C., about 13° C. to about 24° C., about 13° C. to about 23° C., about 13° C. to about 22° C., about 13° C. to about 21° C., about 13° C. to about 20° C., about 13° C. to about 19° C., about 13° C. to about 18° C., about 13° C. to about 17° C., about 13° C. to about 16° C., about 13° C. to about 15° C., and about 13° C. to about 14° C. In some embodiments, the sample is maintained at a temperature range selected from the group consisting of about 14° C. to about 25° C., about 14° C. to about 24° C., about 14° C. to about 23° C., about 14° C. to about 22° C., about 14° C. to about 21° C., about 14° C. to about 20° C., about 14° C. to about 19° C., about 14° C. to about 18° C., about 14° C. to about 17° C., about 14° C. to about 16° C., and about 14° C. to about 15° C. In some embodiments, the sample is maintained at a temperature range selected from the group consisting of about 15° C. to about 25° C., about 15° C. to about 24° C., about 15° C. to about 23° C., about 15° C. to about 22° C., about 15° C. to about 21° C., about 15° C. to about 20° C., about 15° C. to about 19° C., about 15° C. to about 18° C., about 15° C. to about 17° C., and about 15° C. to about 16° C. In some embodiments, the sample is maintained at a temperature range selected from the group consisting of about 16° C. to about 25° C., about 16° C. to about 24° C., about 16° C. to about 23° C., about 16° C. to about 22° C., about 16° C. to about 21° C., about 16° C. to about 20° C., about 16° C. to about 19° C., about 16° C. to about 18° C., and about 16° C. to about 17° C. In some embodiments, the sample is maintained at a temperature range selected from the group consisting of about 17° C. to about 25° C., about 17° C. to about 24° C., about 17° C. to about 23° C., about 17° C. to about 22° C., about 17° C. to about 21° C., about 17° C. to about 20° C., about 17° C. to about 19° C., and about 17° C. to about 18° C. In some embodiments, the sample is maintained at a temperature range selected from the group consisting of about 18° C. to about 25° C., about 18° C. to about 24° C., about 18° C. to about 23° C., about 18° C. to about 22° C., about 18° C. to about 21° C., about 18° C. to about 20° C., and about 18° C. to about 19° C. In some embodiments, the sample is maintained at a temperature range selected from the group consisting of about 19° C. to about 25° C., about 19° C. to about 24° C., about 19° C. to about 23° C., about 19° C. to about 22° C., about 19° C. to about 21° C., and about 19° C. to about 20° C. In some embodiments, the sample is maintained at a temperature range selected from the group consisting of about 20° C. to about 25° C., about 20° C. to about 24° C., about 20° C. to about 23° C., about 20° C. to about 22° C., and about 20° C. to about 21° C. In some embodiments, the sample is maintained at a temperature range selected from the group consisting of about 21° C. to about 25° C., about 21° C. to about 24° C., about 21° C. to about 23° C., and about 21° C. to about 22° C. In some embodiments, the sample is maintained at a temperature range selected from the group consisting of about 22° C. to about 25° C., about 22° C. to about 24° C., and about 22° C. to about 23° C. In some embodiments, the sample is maintained at a temperature range selected from the group consisting of about 23° C. to about 25° C., and about 23° C. to about 24° C. In some embodiments, the sample is maintained at a temperature ranging from about 24° C. to about 25° C.

In some embodiments, the sample is maintained at a temperature selected from the group consisting of about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., and about 25° C.

In some embodiments, the time for storing the extended sperm sample is about 2 hours to 96 hours. In some embodiments, the time for storing the extended sperm sample is at least 12 hours, 13 hours, 14 hours, 15 hours, 16 hours, 17 hours, 18 hours, 19 hours, 20 hours, 21 hours, 22 hours, 23 hours, 24 hours, 36 hours, 48 hours, 60 hours, 72 hours, 84 hours, or 96 hours. In some embodiments, the time for storing the extended sperm sample is greater than 2 hours. In some embodiments, the time for storing the extended sperm sample is at least 12 hours. In some embodiments, the time for storing the extended sperm sample is at least 18 hours. In some embodiments, the time for storing the extended sperm sample is at least 24 hours. In some embodiments, the time for storing the extended sperm sample is at least 36 hours. In some embodiments, the time for storing the extended sperm sample is at least 48 hours. In some embodiments, the time for storing the extended sperm sample is at least 60 hours.

In some embodiments, the method comprises adding a buffer solution to the sperm sample to maintain the pH of the sample. In some embodiments, such buffer solution is selected from the group consisting of 2-(N-morpholino)ethanesulfonic acid (MES), (3-(N-morpholino)propanesulfonic acid) (MOPS), piperazine-N,N′-bis(2-ethanesulfonic acid) (PIPES), acetate, borate, citrate, glycine, bicarbonate, TRIS, phosphate, HEPES, citric acid, and combinations thereof.

In an embodiment, the invention provides a method for storing a sperm sample which comprises diluting the sperm sample with semen extender in a ratio of about 10:1 to 1:10, and storing the extended sample in a sealed, thermally insulating container at a temperature from about 4° C. to about 25° C. for a time span of greater than 2 hours, 4 hours, 8, hours, 12, hours, 18 hours, 24 hours, 36 hours, 48 hours, 60 hours, 72 hours, 84 hours, or 96 hours.

The present invention also provides a collection kit for use with the methods of the present invention, including for use with the Cap-Score™ assay for identifying fertility status of a male individual. In some embodiments, the collection kit comprises one or more of the following: one or more sterile sperm storage containers, transfer pipettes, a semen extender solution, an insulating pouch to store the storage container(s), a cold pack (e.g. KOOLIT® refrigerant) to control the temperature to from about 8° C. to about 20° C., a thermally insulating container to store the sample/insulating pouch and cold pack, and instructions for use. In some embodiments, the kit further comprises a buffer solution to mix with the sperm sample. In some embodiments, such buffer solution is selected from the group consisting of MES, MOPS, PIPES, acetate, borate, citrate, glycine, bicarbonate, TRIS, sodium phosphate, HEPES, citric acid, and combinations thereof.

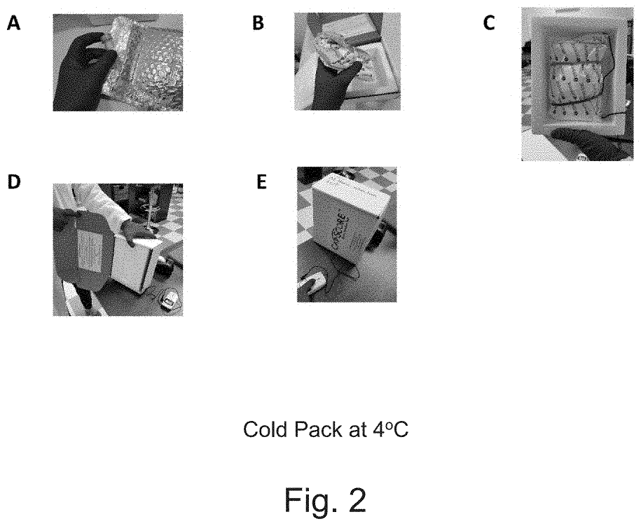

In some embodiments, the method comprises collecting the sperm sample into a collection cup, using a transfer pipette to move a portion of or all of the sample into a 15 mL conical tube, diluting the sperm sample at 1:1 (v/v) ratio with a semen extender, wrapping the conical tube and its contents in an insulated bubble foil pouch, placing the foil pouch-wrapped sample tube into a Styrofoam box, placing a cold pack chilled to 4° C. on top of the bubble foil pouch wrapped sample tube pack, sealing the Styrofoam box, and placing the Styrofoam box into a shipping container (See FIG. 2).

In some embodiments, the method comprises diluting the sperm sample 1:1 (v/v) in a 15 mL conical tube with pre-warmed refrigeration medium-TYB medium (TYB extender; Irvine Scientific; 90129-20×5 mL), inserting the tube into a foil bubble pouch mailer (Therapak; 56362G), placing a cold pack (Therapak; 562200) that was maintained at 4° C. onto the mailer, placing the mailer/cold pack in a polystyrene foam cooler (6^{3/8″}×4^{7/8″}×2^{1/4″} inner diameter with a ¾″ wall thickness), and placing the cooler in a cardboard box (FIG. 2).

In some embodiments, the method comprises collecting the sperm sample into a collection cup, using a transfer pipette to move a portion of or all of the sample into a 15 mL conical tube, diluting the sperm sample 1:1 (v/v) with a semen extender, wrapping the conical tube and its contents with an insulated bubble foil pouch, placing the foil pouch-wrapped sample tube into a Styrofoam box, placing a cold pack chilled to −20° C. on top of the bubble foil pouch wrapped sample tube pack, sealing the Styrofoam box, and placing the Styrofoam box into a shipping container.

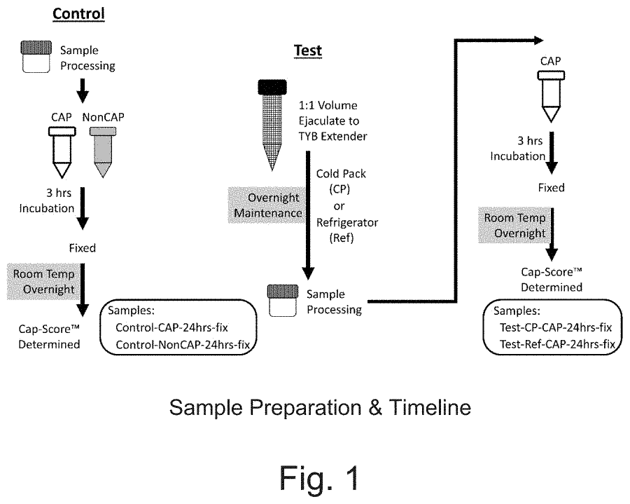

In some embodiments, the collected sperm sample is processed for the Cap-Score™ assay after addition of the semen extender. In some embodiments, the collected sperm sample is processed immediately after collection (without addition of the semen extender) for the Cap-Score™ assay. In some embodiments, processing of the sperm sample comprises one or more steps selected from the group consisting of removing the semen extender, washing the sperm sample, resuspending one or more portions of the sperm sample in a medium, and adding a capacitation agent to one or more portions of the sperm sample to induce capacitation. In some embodiments, processing comprises adding a capacitation buffer or a non-capacitation buffer to a portion of the collected sperm sample (FIG. 1).

In some embodiments, the extender and seminal plasma are separated from the sperm by centrifugation and subsequent washing of the pelleted sperm sample with a medium, for example, mHTF (Irvine Scientific; reference 90126). In some embodiments, the centrifugation speed ranges from about 50 g to about 20,000 g. In some embodiments, the centrifugation speed ranges from about 100 g to about 2000 g. In some embodiments, the centrifugation speed ranges from about 300 g to about 600 g.

In some embodiments, home collected ejaculates within a sealed specimen container (Fisher Scientific, 14-375-462) are liquefied at 37° C. in an air incubator. To remove the extender and/or seminal plasma, 1 mL of sample was layered on to 1 mL of Enhance S-Plus Cell Isolation Media (Vitrolife, reference: 15232 ESP-100-90%). In an embodiment, the samples are centrifuged at 300 g for 10 min., the cell pellets are collected, resuspended in about 4 mL of Modified Human Tubal Fluid medium (mHTF) (Irvine Scientific; reference 90126), and pelleted at 600 g for 10 min.

In some embodiments, the semen extender is separated by centrifugation of the sperm through Enhance S-Plus Cell Isolation Media (Vitrolife; Göteborg, Sweden; catalogue #15232 ESP-100-90%) and the sperm is washed with a medium, for example, modified Human Tubal Fluid medium (mHTF; Irvine Scientific, Santa Ana, Calif.; catalogue #90126). The sperm sample tube is centrifuged at 600 g for 10 minutes. In some embodiments, the sperm cells are re-suspended in a medium, for example, mHTF, with (Cap) and, optionally, without (NonCap) a sperm capacitation agent, such as, 2-hydroxypropyl-β-cyclodextrin (Sigma; St. Louis, Mo.; catalog #C0926) (FIG. 1).

In some embodiments, the sperm cells were separated from the semen extender by centrifugation with Enhance S-Plus Cell Isolation Media (Vitrolife, Englewood, Colo., reference: 15232 ESP-100-90%) at 300 g for 10 minutes. In one embodiment, the sperm cells are collected and re-suspended with approximately 4 mL of human tubal fluid (HTF) (Irvine Scientific, Santa Ana, Calif., reference 90125) or modified human tubal fluid (mHTF) (Irvine Scientific, reference 90126), and centrifuged again at 600 g for 10 minutes. The resultant sperm cell pellet is re-suspended in HTF or mHTF and may be divided into two separate aliquots incubated with (Cap) and, optionally, without (Non-Cap) a capacitation agent. Sperm concentration is adjusted to 10 million sperm cells/ml per tube, and then incubated for 3 hours at 37° C. In some embodiments, sperm cells are incubated at 37° C. in an incubator with 5% CO₂ in HTF containing a bicarbonate buffer. In some embodiments, sperm cells are incubated at 37° C. in an air incubator in mHTF containing a HEPES buffer.

In some embodiments, following incubation with or, optionally, without the capacitation agent, the sperm samples are fixed, packaged and shipped or stored for at least a period of 18 hours before the Cap-Score™ is determined. In another embodiment of the invention, incubation with the capacitation agent occurs after shipment and/or storage for at least a period of 18 hours.

In some embodiments, the isolated sperm cells are subjected to one or more selection processes selected from the group consisting of layering on top of, and centrifugation through a density gradient; layering on top of, and centrifugation through a density gradient followed by collection of the sperm-enriched fraction followed by resuspension and washing; layering on top of, and centrifugation through a density gradient followed by collection of the sperm-enriched fraction and overlaying on top of that a less dense medium into which motile sperm will swim up; and overlaying a less dense medium on top of the sample and allowing motile sperm to swim up into it.

In some embodiments, the sperm cells are counted, and a given number of sperm are placed into containers (such as tubes) and diluted with non-capacitating medium or medium comprising a capacitation agent to achieve desired final concentrations of sperm. In some embodiments, the final desired concentration of sperm is 10 million/mL (final concentration ranges might vary from 250×10³ sperm/mL to 250×10⁶ sperm/mL).

In some embodiments, the medium useful in the present invention (for example, for resuspending sperm, for washing the sperm sample, and/or for incubating the sperm under non-capacitating and capacitating conditions), is a physiological buffered solution. In some embodiments, the medium is selected from the group consisting of human tubal fluid (HTF); modified human tubal fluid (mHTF); Whitten's medium; modified Whitten's medium; KSOM; phosphate-buffered saline; HEPES-buffered saline; Tris-buffered saline; Ham's F-10; Tyrode's medium; modified Tyrode's medium; TES-Tris (TEST)-yolk buffer; or Biggers, and Whitten and Whittingham (BWW) medium. In some embodiments, the medium comprises one or more defined or complex sources of protein. In some embodiments, the protein source is selected from the group consisting of fetal cord serum ultrafiltrate, plasmanate, egg yolk, skim milk, albumin, lipoproteins, and fatty acid binding proteins. In some embodiments, the addition of protein to the medium promotes viability and/or aids in inducing capacitation.

In some embodiments, a sperm sample is subjected to at least one capacitation agent to conduct the Cap-Score™ analysis. In some embodiments, the stimuli for capacitation is one or more of bicarbonate (typically at 20-25 mM, with ranges from 5-50 mM), calcium (typically at 1-2 mM, with ranges from 0.1-10 mM), and/or cyclodextrin (typically at 1-3 mM, with ranges from 0.1-20 mM). In some embodiments, the cyclodextrins are selected from the group consisting of 2-hydroxypropyl-β-cyclodextrin, methyl-β-cyclodextrin, and combinations thereof.

In some embodiments, a capacitating agent or stimulus is selected from the group consisting of bicarbonate ions, calcium ions, mediators of sterol efflux, and combinations thereof. In some embodiments, a mediator of sterol efflux is selected from the group consisting of 2-hydroxypropyl-β-cyclodextrin, methyl-β-cyclodextrin, serum albumin, high density lipoprotein, phospholipids vesicles, fetal cord serum ultrafiltrate, fatty acid binding proteins, liposomes, and combinations thereof. In some embodiments, exposure of a control sample to capacitating or, optionally, non-capacitating conditions can be done in parallel with the test sample.

In some embodiments, incubation temperatures with capacitation agent or stimulus range from about 30° C. to about 40° C., from about 30° C. to about 39° C., from about 30° C. to about 38° C., from about 30° C. to about 37° C., from about 30° C. to about 36° C., from about 30° C. to about 35° C., from about 30° C. to about 34° C., from about 30° C. to about 33° C., from about 30° C. to about 32° C., or from about 30° C. to about 31° C. In some embodiments, the incubation temperature is about 30° C., about 31° C., about 32° C., about 33° C., about 34° C., about 35° C., about 36° C., about 37° C., about 38° C., about 39° C., or about 40° C. In some embodiments, incubation time with a capacitation agent ranges from about 30 minutes to about 18 hours. In some embodiments, the incubation time ranges from about 1 hour to about 4 hours. In some embodiments, the incubation time is about 30 minutes or 0.5 hour. In some embodiments, the incubation time is about 1 hour. In some embodiments, the incubation time is about 2 hours. In some embodiments, the incubation time is about 3 hours. In some embodiments, baseline samples or measurements are taken at the start of the incubation period.

In some embodiments, sperm cells are fixed to assist with visualization of the G_{M1} pattern after incubation with or without a capacitation agent. Fixation is not necessary in order to assess some G_{M1} patterns; however, fixation preserves the sperm cells, allowing for an extended opportunity to visualize the sperm cells and immobilizing the sperm cells. Various fixatives for histological study of spermatozoa are within the purview of those skilled in the art. In some embodiments, suitable fixatives include paraformaldehyde, glutaraldehyde, Bouin's fixative, and fixatives comprising sodium cacodylate, calcium chloride, picric acid, tannic acid and like. In some embodiments, paraformaldehyde, glutaraldehyde or combinations thereof are used.

In some embodiments, the amount of fixation agent is selected from the group consisting of about 0.004% (weight/volume) paraformaldehyde to about 4% (weight/volume) paraformaldehyde, about 0.01% to about 1% (weight/volume) paraformaldehyde, about, 0.005% (weight/volume) paraformaldehyde to about 1% (weight/volume) paraformaldehyde, about 4% paraformaldehyde (weight/volume), about 0.1% glutaraldehyde (weight/volume), and about 5 mM CaCl₂ in phosphate buffered saline.

Preliminary studies observed no difference in Cap-Score™ assay viability, or sperm recovery whether bicarbonate (HCO₃⁻) or HEPES buffered medium was used. The capacitation stimuli consisted of 2-hydroxypropyl-β-cyclodextrin (Sigma, St. Louis, Mo., catalog # C0926). Pilot studies showed that this stimulus was as effective in promoting capacitation in human sperm, as measured with the Cap-Score™, at 3 hours of incubation as albumin was at 6 hours. Following incubation, the samples were fixed with paraformaldehyde (Electron Microscopy Services, catalogue #15712). In some embodiments, the Cap-Score™ assay is performed on the sperm sample as described in the present invention. The Cap-Score™ assay is a proprietary assay developed by Androvia LifeSciences, and described in U.S. patent application Ser. Nos. 15/512,357, 15/387,965, 15/435,875. The Cap-Score™ assay measures differences in G_{M1} distribution patterns in live or fixed sperm cells using affinity binding molecules having specific affinity for the G_{M1} ganglioside. In some embodiments, the affinity molecule is directly linked to a detectable label (such as a fluorophore). In some embodiments, the affinity molecule is detected by a second affinity molecule that has a detectable label on it. In some embodiments, a labeled (such as fluorescent labeled) cholera toxin b subunit is used to obtain a G_{M1} distribution pattern. In some embodiments, the amount of the cholera toxin b subunit fluorophore label used is from about 0.01 to about 10.0 μg/ml. In some embodiments, the amount of the cholera toxin b subunit fluorophore label used is about 0.01, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.4, 2.6, 2.7, 2.7, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 6.0, 7.0, 8.0, 9.0, or 10.0 μg/mL. In some embodiments, the amount is about 2 μg/mL. In some embodiments, a labeled antibody to the cholera toxin b subunit is used to visualize the G_{M1} distribution pattern. In some embodiments, a labeled secondary antibody that binds to either the primary antibody that binds directly to G_{M1} or to the primary antibody that binds to the b subunit of cholera toxin is used. In some embodiments, other detectable labels useful in the present invention are selected from a radionuclide, an enzyme, a fluorescent agent or a chromophore. In some embodiments, labeling (or staining) and visualization of G_{M1} distribution in the sperm sample according to the present invention is carried out by standard techniques known in the art, including, but not limited to use of polyclonal and monoclonal antibodies, generation of peptide mimics of relevant epitopes of the G_{M1} molecule, or mimicking binding with a small molecule.

As more fully discussed in U.S. patent application Ser. Nos. 15/512,357, 15/387,965, 15/435,875, the Cap-Score™ is determined by comparing the G_{M1} distribution patterns of capacitated and non-capacitated sperm samples and assigning a Cap-Score™ based on the differences in those patterns that reflect the percentage of sperm that respond to the stimulus for capacitation. For human sperm, several different G_{M1} patterns were reported. These patterns are designated as INTER (intermediate), APM (Acrosomal Plasma Membrane), AA (apical acrosome), PAPM (Post Acrosomal Plasma Membrane), AA/PA (apical acrosome/post acrosome), ES (equatorial segment), DIFF (diffuse), and Lined Cell. The fertility threshold is the value of AA and/or APM at which the fertility of a population ceases to substantially increase for increasing levels of AA and/or APM. Individuals may be designated as “infertile,” “sub-fertile,” or “fertile” based on the individual's level of AA and/or APM (See Example 1 of U.S. application Ser. No. 15/512,357). In an embodiment, the percentage of cells having patterns indicative of having capacitated (e.g., AA+APM) was assessed.

The present invention also provides a collection kit for use with the methods described herein, including for use with the Cap-Score™ assay for identifying fertility status of a male individual. In some embodiments, the collection kit comprises one or more of the following: one or more sterile sperm storage containers, transfer pipettes, a semen extender solution, an insulating pouch to store the storage container, a cold pack to control the temperature at about 4° C. to about 25° C., a thermally insulating container to store the sample/insulating pouch and cold pack, and instructions for use. In some embodiments, the kit further comprises a buffer solution to mix with the sperm sample. In some embodiments, the buffer solution is selected from the group consisting of MES, MOPS, PIPES, acetate, borate, citrate, glycine, bicarbonate, TRIS, sodium phosphate, HEPES, citric acid, and combinations thereof.

In some embodiments, the kit comprises instructions for use, labels and containers/bags/pouches useful for shipping, storage and identification purposes. In some embodiments, the kit comprises a foil pouch, a biohazard bag with absorbent material for mailing a sperm sample, a re-sealable bag with absorbent, and a foam tube place holder. In some embodiments, the container may be glass, plastic or other durable material.

In some embodiments, the kit comprises one or more of the following: capacitating media, non-capacitating media, fixative composition, reagents for determining G_{M1} staining patterns, comparison charts, predetermined criteria, representations of G_{M1} patterns for comparison, or threshold values.

In some embodiments, the kit further comprises cell isolation media (such as, for example, Enhance S-Plus Cell Isolation Media, 90% from Vitrolife, reference: 15232 ESP-100-90%).

In some embodiments, the kit can further comprise large orifice pipet tips (for example, 200 μL large orifice tip, USA scientific, catalogue #1011-8400) or large orifice transfer pipets (for example, General Purpose Transfer Pipets, Standard Bulb reference number: 202-20S, VWR catalogue #14670-147).

In some embodiments, the kit can further comprise collection and/or storage tubes, for example, 1.5 mL tubes (for example, USA Scientific, catalogue #14159700) and/or 120 mL specimen collection cups (Fisher Scientific, 14375462). In some embodiments, one or more of the collection tubes comprises cyclodextrin to stimulate capacitation. In some embodiments, the capacitation agent, such as cyclodextrin, is packed separately in the kit.

In some embodiments, the kit further comprises density gradient materials and/or instructions to remove the seminal plasma or the semen extender from the sperm sample.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Materials and Methods for all Examples.

The following examples demonstrate the use of a collection kit where the collected sperm samples were maintained for a prolonged period of time, e.g. more than 2 hours.

Sperm samples were collected by male donors into a 120 ml specimen container, allowed to liquefy for at least 15 minutes but no longer than 2 hours and then transferred into a 15 mL conical tube using a large orifice transfer pipette. The sperm samples were diluted 1:1 with semen extender solution and placed in an insulated box containing a cold pack to maintain a temperature from about 4 to about 25° C. Maintenance of samples at reduced temperature was averaged at least 12 hours.

Following overnight maintenance at reduced temperatures, the sperm cells were remove from the seminal plasma and extender. This was accomplished by layering 1 mL of semen/extender on to of 1 mL of Enhance S-Plus Cell Isolation Media in a 15 mL conical tube. Sufficient conical tubes were used to isolate sperm from the entire volume of semen/extender. The tube containing the semen/extender and cell isolation media was centrifuged at 300 g for 10 minutes. The seminal plasma and extender were removed from all tubes and the bottom about 1 mL fractions were transferred to a new 15 mL tube and then resuspended in 4 mL of mHTF. The resuspended sperm were centrifuged at 600 g for 10 minutes. The supernatant was removed and the pellet of sperm cells were resuspended in ˜0.25 mL of mHTF. The washed sperm cells were then evaluated for concentration and motility. The sperm cells were then divided into two tubes, such that the final volume of each tube was 300 μL, and the final concentration of sperm was 10,000,000/mL. The first tube contained 300 μL of mHTF (non-capacitating conditions) and the second tube contained 300 μL of mHTF plus 2-hydroxypropyl-β-cyclodextrin at a final concentration of 3 mM (capacitating condition). Sperm cells were incubated for 3 hours at 37° C.

At the end of the incubation period, the contents of each tube were mixed gently, and 33 μL of 1% (weight/volume) paraformaldehyde was added to achieve a final concentration of 0.1%. In some embodiments, 0.1% (weight/volume) paraformaldehyde was added to achieve a final concentration of 0.01%. These tubes were mixed gently and maintained overnight at room temperature. The next day, 1 μL of 0.5 mg/mL cholera toxin b subunit conjugated with Alexa Fluor 488 was added. The contents of the two tubes were again mixed gently and allowed to set for an additional 10 minutes at room temperature. From each tube, 5 μL was removed and placed on a glass slide for evaluation by fluorescence microscopy.

Following shipping, processing, incubation, and overnight fixation, samples were labeled with 2 μg/mL of Alexa Fluor 488-conjugated cholera toxin beta subunit (Thermo Fisher Scientific; Waltham, Mass.; catalog #C34775). After ten minutes, 5 μL of the labeled sperm replaced on a microscope slide, overlaid with a coverslip (22×22 mm no. 1), and moved to an imaging station.

Imaging was performed on Nikon Eclipse NI-E microscopes equipped with CFI60 Plan Apochromat Lambda 20× Objectives; C-FL AT GFP/FITC Long-Pass Filter Sets; Hamamatsu ORCA-Flash 4.0 cameras; H101F-ProScan III Open Frame Upright Motorized H101F Flat Top Microscope Stages; and 64-bit imaging workstations running NIS Elements software (Nikon; Melville N.Y.).

The proportion of sperm within a sample having undergone capacitation was determined and reported as the Cap-Score (# of sperm with patterns associated with capacitation/(# of sperm with patterns associated with capacitation+number of sperm with other patterns)). All readings were performed according to validated methods (Moody, Cardona et al. 2017), and consistent with Clinical Laboratory Improvement Amendments (CLIA)-, College of American Pathologists (CAP)- and Clinical Laboratory Evaluation Program (CLEP)-approved best practices for quality control and assurance. Briefly, if sufficient sperm were present in the sample, at least 150 total patterns were determined for each condition. If insufficient cells were available, a minimum of 100 patterns is necessary to compute Cap-Score™. Otherwise, the samples were rejected.

### Example 1. Effects of Semen Extender and Temperature on the Sperm Cell Viability During Storage/Transporting for a Period of 24 Hours

In general, semen samples were collected and split. Half served as a control (Control) and the other half served as the test (Test). The Control samples were processed, and two treatments were created. One with (Control-CAP) and one without (Control-NonCAP) a capacitation stimulus. The samples were incubated, fixed and then maintained overnight before being evaluated for Cap-Score (Control-CAP-24 hrs-fix and Control-NonCAP-24 hrs-fix). The Test samples were diluted 1:1 with refrigeration medium (TYB Extender) and in one set of experiments maintained overnight with a Cold Pack (Test-CP) and in another maintained in a Refrigerator (Test-Ref). Following overnight maintenance in the TYB extender and at a reduced temperature, the test samples were processed, and a treatment created containing a capacitation stimuli (Test-“CP, or Ref”-CAP). The samples were incubated, fixed, maintained for a second overnight and then Cap-Score was determined (Test-“CP or Ref”-CAP-24 hrs-fix) (FIG. 1).

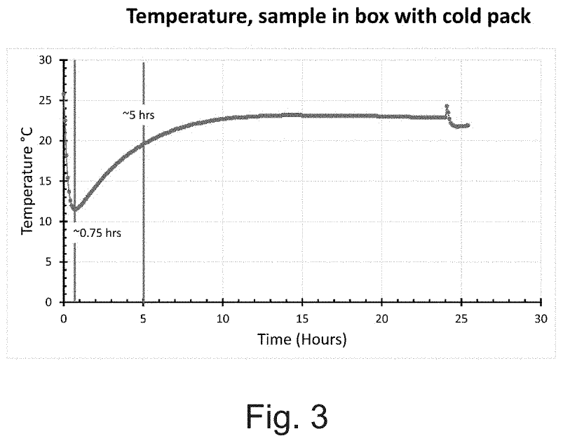

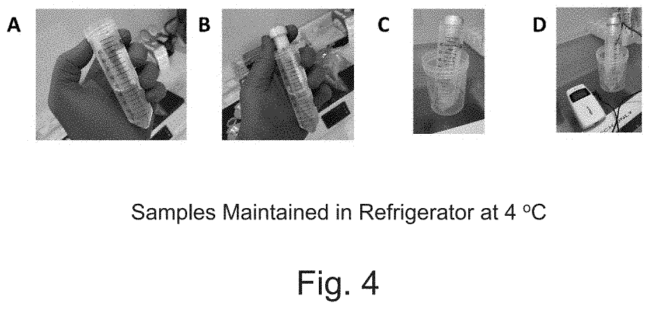

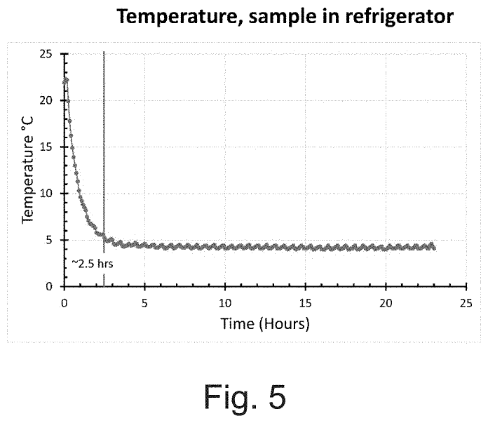

Semen samples for Samples 1-8 in Table 1 below were collected by masturbation and allowed to liquefy for up to 2 hrs (Moody, Cardona et al. 2017). Following liquefaction, the samples were split (FIG. 1). Half served as a control and was processed as normal for Cap-Score™ (Control). The other half served as the test treatment (Test) and was diluted 1:1 with pre-warmed refrigeration medium (TYB extender; Irvine Scientific; 90129-20×5 mL [176 mM 2-[[1,3-dihydroxy-2-(hydroxymethyl)propan-2-yl]amino]ethanesulfonic acid [TES], 80 mM 2-Amino-2-(hydroxymethyl)propane-1,3-diol [Tris], 9 mM dextrose, 10 μL gentamicin sulfate, 20% v/v heat-inactivated egg yolk]). To simulate the time and potential environments encountered during the shipment of samples from a patient's home to a laboratory for processing, two different overnight maintenance protocols were evaluated on the Test samples. First, some test samples were maintained in a Styrofoam box with a cold pack chilled to 4° C. (Test-CP; FIG. 2; n=5). A 15 mL conical tube containing the sample and extender was placed in an insulated foil pouch (FIG. 2A). A cold pack, at 4° C. was wrapped with the pouch and secured with rubber bands (FIG. 2B). The sample, pouch and cold pack were placed into a Styrofoam box (FIG. 2C). The sample, pouch, cold pack and Styrofoam box were then placed into a cardboard box and maintained overnight (FIG. 2D). A temperature probe was inserted into the pouch to obtain temperature readings every 5 minutes (FIG. 2E). A representative temperature profile is shown in FIG. 3. Second, some Test samples were maintained in a refrigerator at 4° C. (Test-Ref; FIG. 4; n=3). About 20 mL of water was added to a 50 mL tube (FIG. 4A). The 15 mL conical tube with the diluted sample was placed into the water (FIG. 4B). The conical tubes were placed in a collection cup and put in a refrigerator maintained at 4° C. (FIG. 4C). A temperature probe was inserted into the water bath to obtain temperature readings every 5 minutes (FIG. 4D). A representative temperature profile is shown in FIG. 5.

Following liquefaction for the Control and after overnight maintenance for the Test samples, the sperm were removed from the seminal plasma and or TYB extender by centrifugation through Enhance S-Plus Cell Isolation Media (Vitrolife; Göteborg, Sweden; catalogue #15232 ESP-100-90%) and washed with modified Human Tubal Fluid medium (mHTF; Irvine Scientific, Santa Ana, Calif.; catalogue #90126, 97.8 mM NaCl, 4.69 mM KCl, 0.20 mM MgSO₄, 0.37 mM KH₂PO₄, 2.04 mM CaCl₂, 4 mM NaHCO₃, 21 mM 4-(2-hydroxyethyl)-1-piperazineethanesulfonic acid [HEPES], 2.78 mM C6H1206, 0.33 mM sodium pyruvate, 21.4 mM sodium lactate, 10 μg/ml gentamicin, 5 mg/L phenol red). The sperm were resuspended in mHTF either without (NonCAP) or with (CAP) 2-hydroxypropyl-O-cyclodextrin (Sigma; St. Louis, Mo.; catalog #C0926) to promote capacitation (Moody, Cardona et al. 2017). After three hours of incubation, the samples were fixed. For the Control samples, NonCAP and CAP Cap-Scores were determined approximately 24 hours after fixation (Control-CAP-24 hrs-fix and Control-NonCAP-24 hrs-fix). For the Test samples, Cap-Scores were determined after an additional overnight maintenance at room temperature (Test-CP-CAP-24 hrs-fix or Test-Ref-CAP-24 hrs-fix). It is anticipated that the additional overnight in fix is needed for the sample to reach maximum capacitation levels, as exposure of the sperm to TYB could be limiting Capacitation (Ostermeier, Cardona et al. 2018).

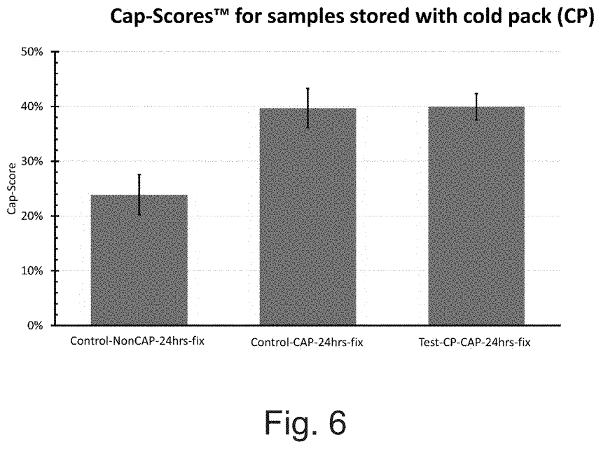

As shown in FIG. 6, semen samples were collected and split. Half served as a control (Control) and half as the test (Test). The control samples were allowed to liquefy and then processed to create samples without (Control-NonCAP) and with (Control-CAP) capacitation stimuli. Following incubation, the control samples were fixed and maintained overnight before being evaluated for Cap-Score (Control-NonCAP-24 hrs-fix and Control-CAP-24 hrs-fix). In this set of experiments, the test samples were first extended in refrigeration media and then maintained overnight with a Cold Pack (Test-CP). These samples were then processed, incubated, fixed overnight and Cap-Score was determined (Test-CP-CAP-24 hrs-fix). Since ejaculates (n=5) were split, paired t-tests were used for pre-planned comparisons. As expected, the Control-CAP-24 hrs-fix was greater than the Control-NonCAP-24 hrs-fix (p<0.001). No difference was observed between the Control-CAP-24 hrs-fix and Test-CP-CAP-24 hrs-fix (p=0.865).

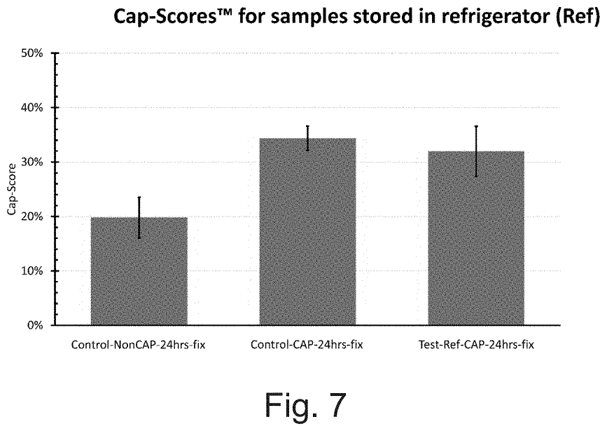

As shown in FIG. 7, semen samples were collected and split. Half served as a control (Control) and half as the test (Test). The control samples were allowed to liquefy and then processed to create samples without (Control-NonCAP) and with (Control-CAP) capacitation stimuli. Following incubation, the control samples were fixed and maintained overnight before being evaluated for Cap-Score (Control-NonCAP-24 hrs-fix and Control-CAP-24 hrs-fix). The test samples were first diluted in refrigeration media and then maintained overnight in a refrigerator (Test-Ref). These samples were then processed, incubated, fixed overnight and Cap-Score was determined (Test-Ref-CAP-24 hrs-fix). Since ejaculates (n=3) were split, paired t-tests were used for pre-planned comparisons. As expected, the Control-CAP-24 hrs-fix was greater than the Control-NonCAP-24 hrs-fix (p=0.021). No difference was observed between the Control-CAP-24 hrs-fix and Test-Ref-CAP-24 hrs-fix (p=0.482).

Cap-Scores were greater for the Control-CAP-24 hrs-fix samples when compared to Control-NonCAP-24 hrs-fix in both the cold pack (FIG. 6) and refrigerator (FIG. 7) experiments (p<0.05). This demonstrates a good capacitation response in both experiments using the standard approach. Similar Cap-Score values were observed between the Control-CAP-24 hrs-fix and Test-CP-CAP-24 hrs-fix (FIG. 6), indicating that ejaculates can be maintained overnight in TYB extender and processed the following day. Similarly, in the refrigerator experiment, the Cap-Score of the Control-CAP-24 hrs-fix was not different from the Test-Ref-CAP-24 hrs-fix samples (FIG. 7). This observation further substantiates that ejaculates can be maintained overnight in refrigeration medium and processed the following day for Cap-Score.

The temperature profiles generated in the cold pack chilled to 4° C. (FIG. 3) and refrigerator at 4° C. (FIG. 5) experiments appear unique from one another. With the cold pack set up, the temperature dropped to about 11° C. in about 0.75 hours and then rebounded to room temperature (about 20° C.) in about 4.5 hrs. Thus, the samples were maintained at room temperature for about 19 hrs. In contrast, with the refrigerator set up, the temperature dropped to about 4° C. in about 2.5 hrs and stayed there until the samples were processed the next day. These results suggested that sperm in TYB extender can be maintained overnight in a broad range of temperatures and then processed to obtain Cap-Scores that a comparable to those generated using standard processing.

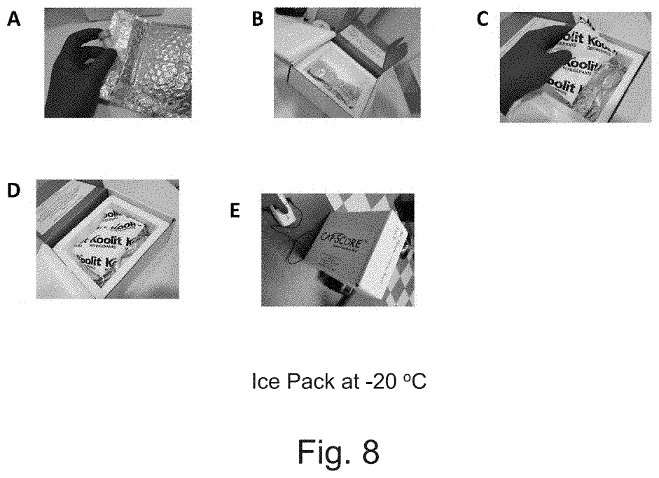

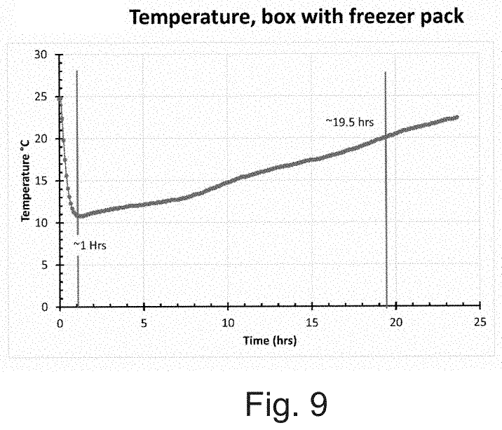

To determine if a more moderate temperature profile could be generated, one that would prevent the samples from reaching either of the extreme environments documented above, mock samples were placed in a box with an ice pack chilled to −20° C. (FIG. 8). A 15 mL conical tube containing the sample and extender was placed in an insulated foil pouch (FIG. 8A). The foil pouch was wrapped around the sample and rubber bands secured the pouch before being placed into a Styrofoam box (FIG. 8B). An ice pack equilibrated to −20° C. was placed on top of the secured pouch and sample (FIG. 8C). The sample, pouch, ice pack and Styrofoam box were placed into a cardboard box and maintained overnight (FIG. 8D). A temperature probe was inserted into the pouch to obtain temperature readings every 5 minutes (FIG. 8E). A representative profile is shown in FIG. 9. Indeed, when using the icepack setup, the samples reached about 12° C. in about 1 hr and did not return to room temperature until about 19.5 hrs had elapsed.

Together, these data support the development of an at home Cap-Score™ collection kit. This kit will allow patients to conveniently provide ejaculates at home and ship them overnight to a centralized laboratory for processing.

The Cap-Score™ Assays were performed on extended human sperm samples subjected to various temperature treatment during storage to determine the effects of the extender solution and the temperature control on the capacitation capability of the sperm cells. The Cap-Score™ Assay results are summarized in Table 1.

TABLE 1
Extender and Temperature Effects on Sperm Capacitation Score

| | Control- | | | |
|---|---|---|---|---|
| | NonCAP- | Control-CAP- | Test-CP-CAP- | Test-Ref-CAP- |
| | 24 hrs-fix | 24 hrs-fix | 24 hrs-fix¹ | 24 hrs-fix² |
| Example 1 | 35.6% | 50.3% | 45.75% | — |
| Example 2 | 24.5% | 45.1% | 45.3% | — |
| Example 3 | 27% | 39% | 39% | — |
| Example 4 | 27% | 39% | — | 41% |
| Example 5 | 14.4% | 33.1% | 34.4% | |
| Example 6 | 14.4% | 33.1% | | 25.6% |
| Example 7 | 18.1% | 31.3% | 35.1% | — |
| Example 8 | 18.1% | 31.3% | — | 29.3% |

¹Test-CP-CAP-24 hrs-fix time = 24 hrs; the sperm sample extended with TYB and stored inside a Styrofoam box chilled with a cold pack chilled to 4° C.
²Test-Ref-CAP-24 hrs-fix: time = 24 hrs; the sperm sample extended with TYB and stored inside a refrigerator at 4° C.

With regard to the extended semen sample maintained overnight with a cold pack (Test-CP), the control samples were allowed to liquefy and then processed to create samples without (Control-NonCAP) and with (Control-CAP) capacitation stimuli. Following incubation, the control samples were fixed and maintained overnight before being evaluated for Cap-Score (Control-NonCAP-24 hrs-fix and Control-CAP-24 hrs-fix). In this set of experiments, the test samples were first extended in refrigeration media and then maintained overnight with a cold pack (Test-CP). These samples were then processed, incubated, fixed overnight and Cap-Score was determined (Test-CP-CAP-24 hrs-fix). Since ejaculates (n=5) were split, paired t-tests were used for pre-planned comparisons. As expected, the Control-CAP-24 hrs-fix was greater than the Control-NonCAP-24 hrs-fix (p<0.001). No difference was observed between the Control-CAP-24 hrs-fix and Test-CP-CAP-24 hrs-fix (p=0.865) (FIG. 6).

With regard to the semen sample stored in refrigerator, the control samples were allowed to liquefy and then processed to create samples without (Control-NonCAP) and with (Control-CAP) capacitation stimuli. Following incubation, the control samples were fixed and maintained overnight before being evaluated for Cap-Score™ (Control-NonCAP-24 hrs-fix and Control-CAP-24 hrs-fix). The test samples were first diluted in refrigeration media and then maintained overnight in a refrigerator (Test-Ref). These samples were then processed, incubated, fixed overnight and Cap-Score™ was determined (Test-Ref-CAP-24 hrs-fix). Since ejaculates (n=3) were split, paired t-tests were used for pre-planned comparisons. As expected, the Control-CAP-24 hrs-fix was greater than the Control-NonCap-24 hrs-fix (p=0.021). No difference was observed between the Control-CAP-24 hrs-fix and the Test-Ref-CAP-24 hrs-fix (p=0.482) (FIG. 7).

The results in Table 1 demonstrated that there is no effective difference between the control samples and those maintained overnight at reduced temperatures. The tolerance of the sperm cell capacitation capability to the TYB extender (Irvine Scientific, catalog #90129) at a chilled temperature for a prolonged period reduced the need of collecting sperm sample at the clinical Cap-Score™ testing lab with the oversight of a practicing physician. Put in another way, the tolerance of the sperm cell capacitation capability to the extender at a temperature between 8° C. and 25° C. enables the patient to collect the sperm sample at home and ship the extended sperm sample overnight to the clinical Cap-Score™ testing lab overnight. The convenience of home collection of sperm sample greatly improves the Cap-Score assay availability without loss of accuracy or efficiency.

### Example 2. Impact of TEST Yolk Buffer (TYB) and Cooling on the Ability of Human Sperm to Capacitate, as Determined by Cap-Score™

This study is to evaluate the impact of TEST (TES and Tris) yolk buffer and cooling on the ability to extend the time for sperm capacitation.

Semen samples 1-3 in Table 2 below were collected, liquefied and split into control and test samples according to the procedures set forth in Example 1 above (FIG. 1, Moody, Cardona et al. 2017). Half served as a control and were processed as normal for Cap-Score™ (Control) and the other half served as the test sample (Test). Test samples were extended in TYB medium (Irvine Scientific; 90129-20×5 mL [176 mM 2-[[1,3-dihydroxy-2-(hydroxymethyl)propan-2-yl]amino]ethanesulfonic acid [TES], 80 mM 2-Amino-2-(hydroxymethyl)propane-1,3-diol [Tris], 9 mM dextrose, 10 μL gentamicin sulfate, 20% v/v heat-inactivated egg yolk]). The extended semen test samples were diluted at a volume ratio of semen:TYB of 1:1 (n=5), 1:6 (n=7), or 8:5 (n=5). TYB extended test semen samples 1-3 were stored for overnight (i.e., about 18 hours to about 24 hours) in a sealed insulated Styrofoam box with a cold pack chilled to −20° C.

First, the chilled sample has been warmed back to room temperature following the overnight storage. Second, the samples were washed, exposed to non-capacitating (NC) or capacitating (CAP) conditions for 3 hrs, and then were fixed overnight before Cap-Score determination. Cap-Score for the test samples 1-3 in Table 2 was determined following the assay procedures set forth in Example 1 above. Test-samples were compared to controls using paired t-tests.

In all experiments, Cap-Score was greater for control-CAP when compared to control-NC (p<0.05). No significant differences were observed between the control-CAP and the test-CAP for any dilution (1:1 ratio: 39.7±4% vs 40.0±2%; p=0.87; 1:6 ratio: 32.0±4% vs 34.0±3%; p=0.33; 8:5 ratio: 36.0±2% vs 34.2±1%; p=0.5). The Cap-Score™ test results are summarized in Table 2 below.

TABLE 2
Impact of TYB and Cooling on Sperm Capacitation Score

| | volume | | | | |
|---|---|---|---|---|---|
| | ratio of | | | | |
| | semen:TEST | Control-CAP- | Test-CAP- | Number of | |
| Entry | TYB | overnight | overnight | repeats | p |
| 1 | 1:1 | 39.7 ± 4% | 40.0 ± 2% | 5 | 0.87 |
| 2 | 1:6 | 32.0 ± 4% | 34.0 ± 3% | 7 | 0.33 |
| 3 | 8:5 | 36.0 ± 2% | 34.2 ± 1% | 5 | 0.5 |

According to the results in Table 2, good capacitation responses were observed in the controls for all experiments, suggesting proper stimulus by the CAP conditions. The volume ratios of semen: TYB were chosen to mimic typical ejaculate volumes, such that a constant volume of extender could potentially be utilized in an at home semen collection kit that maintains sperm capacitation ability. Addition of a fixed volume of TYB to varying ejaculate volumes would limit user input. Similar Cap-Score values between the control-CAP and test-CAP, no matter the ratio, indicates that ejaculates can be maintained overnight in varying concentrations of TYB with minimal impact on next-day function.

This study showed that TYB could prolong the fertilization capacity of sperm. TYB can be used as an effective semen extender for the home collection kit as described herein.

Overall, analysis of variance (ANOVA) was used to compare CAP-Score™ and concentration values among all experiments (1:1 cold pack, 1:1 refrigerator, 1:6 ice pack, 8:5 ice pack, 1:1 shipping incubator and 1:1 shipping freezer). No overall effect of experiment was observed for the control or test treatments. To ensure that samples are maintained in the temperature ranges tested, two temperature monitors are included in the at home collection kits to identify samples that reach −5° C. (Telatemp—Cold Snap CT −5° C.; see 1:1 shipping freezer data) and those that go above 20° C. for more than 1 hour (Telatemp—Warm Mark 3TM+20° C.; see 1:1 cold pack data).

At home, sample collection could lessen the burden of processing samples at clinics with limited resources. It could also encourage pursuit of workup by men whose main barrier is privacy in producing samples at clinics or bringing them to clinics. It could also broaden the geographical availability of sperm function tests to those living far from clinics, and reduce financial burdens associated with travel and time away from work.

We claim:

1. A method for identifying fertility status of a human male comprising the steps:
a) obtaining a sperm sample from a human male;
b) introducing into the sperm sample a fixed volume of a semen extender solution at a dilution volume ratio to give an extended sperm sample;
c) maintaining the extended sperm sample at a temperature range of about 4° C. to about 25° C. for an extended time span of greater than 2 hours;
d) performing a Cap-Score assay on the extended sperm sample of step (c) to determine the Cap-Score; and
(e) determining fertility status of the human male.

2. The method of claim 1, wherein the resulting Cap-Score for the extended sperm sample from the Cap-Score assay is not significantly different from a Cap-Score resulting from a similarly processed fresh sample from the same individual.

3. The method of claim 1, wherein the temperature range is about 4° C. to about 20° C.

4. The method of claim 3, wherein the temperature range is about 8° C. to about 10° C.

5. The method of claim 1, wherein the semen extender solution comprises a buffer selected from the group consisting of bicarbonate buffer, citrate buffer, hydroxymethylaminomethane (TRIS) buffer, TRIS/citric acid buffer, TRIS/citrate buffer, 4-(2-hydroxyethyl)-1-piperazineethanesulfonic acid (HEPES) buffer, HEPES/TRIS buffer, N′-tris (hydroxymethyl)methyl-2-aminoethane (TES) and hydroxymethylaminomethane (TRIS) buffer (TES/TRIS buffer), and a combination thereof.

6. The method of claim 5, wherein the buffer is TES/TRIS buffer.

7. The method of claim 1, wherein the semen extender solution comprises a protein selected from the group consisting of albumin, fetal cord serum ultrafiltrate, plasmanate, egg yolk, skim milk, lipoprotein, fatty acid binding protein, and a combination thereof.

8. The method of claim 7, wherein the protein is egg yolk.

9. The method of claim 6, wherein the semen extender solution further comprises an antibiotic.

10. The method of claim 9, wherein the antibiotic is gentamicin.

11. The method of claim 1, wherein the Cap-Score assay comprises the steps of (i) separating sperm cells from the sperm sample and the semen extender solution, (ii) resuspending a first portion of the sperm cells into a buffering system with capacitation stimuli (Cap) and a second portion of the sperm cells into a buffering system without capacitation stimuli (non-Cap), and (iii) incubating the resulting cell suspensions in capacitation and non-capacitation buffering systems for 3 hours at 37° C.

12. The method of claim 1, wherein the dilution volume ratio for the sperm sample to the semen extender solution is about 10:1 to about 1:10.

13. The method of claim 1, wherein the dilution volume ratio for the sperm sample to the semen extender solution is about 1:1.

14. The method of claim 1, wherein the sperm sample is stored in a plastic tube with conical bottom and seal cap.

15. The method of claim 1, wherein the plastic tube is selected from the group consisting of polypropylene, polystyrene, polyethylene terephthalate (PET), low-density polyethylene (LDPE), polyallomer (PA), and polycarbonate (PC).

16. The method of claim 1, wherein the extended time span is greater than 2 hours to about 24 hours.

17. The method of claim 1, wherein the extended time span is at least 18 hours.

18. The method of claim 1, wherein the method further comprises using a sperm sample collection kit including: one or more sperm storage containers, transfer pipettes, an insulating pouch, a cold pack, a thermally insulating container, and a semen extender solution.

19. A method for identifying fertility status of a human male individual comprising the steps:
a) obtaining a sperm sample from a human male;
b) introducing into the sperm sample a fixed volume of a semen extender solution at a dilution volume ratio to give an extended sperm sample;
c) cooling the extended sperm sample of step (b) to reach a temperature of about 8° C. to about 10° C. inside a thermally insulating container to provide a chilled extended sperm sample,
d) maintaining the chilled extended sperm sample at a temperature range of about 8° C. to about 25° C. for an extended time span; wherein the temperature is maintained by packing the extended sperm sample in a thermal insulating pouch with a cold pack chilled to a defined temperature; and
e) performing a Cap-Score assay, for determining the distribution of ganglioside G_{M1} patterns resulting from capacitation, on the extended sperm sample of step (d) and determining fertility status of the human male.

20. The method of claim 19, wherein the cooling step (c) takes place over a period of about one hour.

21. The method of claim 19, wherein the extended time span in step (d) is at least 18 hours.

22. The method of claim 20, wherein the cold pack is chilled to 4° C.

23. The method of claim 20, wherein the cold pack is chilled to −20° C.

24. The method of claim 19, wherein the method further comprises using a sperm sample collection kit including: one or more sperm storage containers, transfer pipettes, an insulating pouch, a cold pack, a thermally insulating container, and a semen extender solution.

25. A kit for determination of male fertility status comprising one or more sperm storage containers, transfer pipettes, an insulating pouch, a cold pack, a thermally insulating over-all container, a semen extender solution, an agent for stimulating capacitation, capacitating media, non-capacitating media, fixative reagents, and reagents for determining distribution of G_{M1} patterns.
